(19) United States
(12) Patent Application Publication
(10) Pub. No.: US 2022/0152064 A1
(43) Pub. Date: May 19, 2022

(54) BENZOAZEPINE COMPOUND-CONTAINING PHARMACEUTICAL COMPOSITION

(71) Applicant: OTSUKA PHARMACEUTICAL CO., LTD., Tokyo (JP)

(72) Inventors: Tomihiko CHIHARA, Osaka-shi (JP); Shingo UNO, Osaka-shi (JP)

(73) Assignee: OTSUKA PHARMACEUTICAL CO., LTD., Tokyo (JP)

(21) Appl. No.: 17/439,061

(22) PCT Filed: Mar. 27, 2020
(86) PCT No.: PCT/JP2020/013935

§ 371 Date: Sep. 14, 2021

(30) Foreign Application Priority Data
Mar. 28, 2019 (JP) 2019-064357

(51) Int. Cl. A61K 31/675

(52) U.S. Cl. CPC A61K 31/675

(57) ABSTRACT

A means for reducing side effects of tolvaptan is provided. Specifically, the means provides a pharmaceutical composition comprising a compound represented by Formula (1):

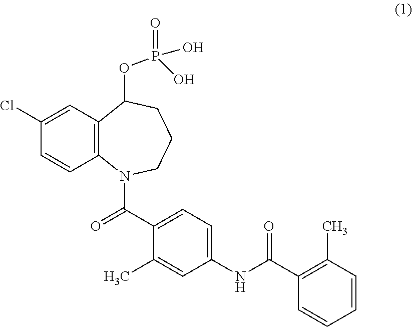 [SMILES: Cc1ccccc1C(=O)Nc1ccc(C(=O)N2CCCC(OP(=O)(O)O)c3cc(Cl)ccc32)c(C)c1]

or a metal salt thereof,
wherein the pharmaceutical composition is used such that the compound represented by Formula (1) or a metal salt thereof is transvascularly administered in an amount of 4 to 20 mg over a period of 10 minutes or more.

### TECHNICAL FIELD

The present disclosure relates to a benzoazepine compound-containing pharmaceutical composition etc. All of the documents disclosed herein, including the following prior art documents (Patent Literature (PTL) and Non-Patent Literature (NPL)), are incorporated herein by reference in their entirety.

### BACKGROUND ART

Tolvaptan, which is a benzoazepine compound, has vasopressin V2 receptor antagonistic activity, and is used as a diuretic etc. The following Formula (2) shows the structural formula of tolvaptan.

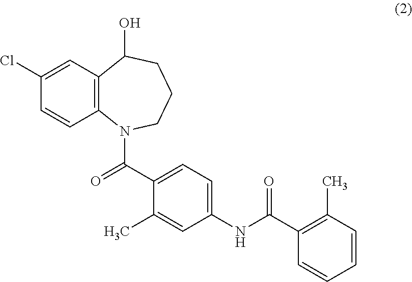 [SMILES: Cc1ccccc1C(=O)Nc1ccc(C(=O)N2CCCC(O)c3cc(Cl)ccc32)c(C)c1]

However, tolvaptan is poorly water-soluble, and there are many restrictions in terms of dosage form, administration route, and the like. Thus, research and development have been conducted on a tolvaptan prodrug that is water-soluble. For example, PTL 1 proposes a tolvaptan prodrug having excellent water solubility.

### CITATION LIST

### Patent Literature

### [PTL 1]

WO2007/074915

### SUMMARY OF INVENTION

### Technical Problem

Although not life-threatening, tolvaptan and tolvaptan prodrugs, when administered, can cause side effects (in particular, side effects on the skin or subcutaneous tissue); thus, reducing these side effects has been demanded.

### Solution to Problem

The present inventors discovered a possibility such that the administration of a specific tolvaptan prodrug at a specific rate can reduce side effects, and made further improvements.

For example, the present disclosure encompasses the subject matter presented in the following items.

Item 1. A pharmaceutical composition comprising a compound represented by Formula (1):

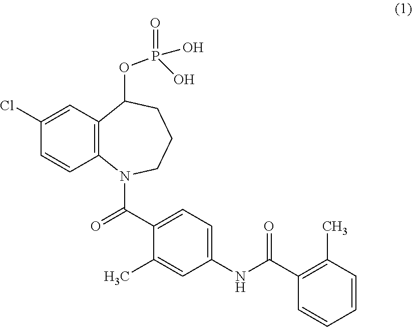 [SMILES: Cc1ccccc1C(=O)Nc1ccc(C(=O)N2CCCC(OP(=O)(O)O)c3cc(Cl)ccc32)c(C)c1]

or a metal salt thereof,
wherein the pharmaceutical composition is used such that 4 to 20 mg of the compound represented by Formula (1) or a metal salt thereof is transvascularly administered over a period of 10 minutes or more.

Item 2. The pharmaceutical composition according to Item 1, which is used such that the compound represented by Formula (1) or a metal salt thereof is administered at an average rate of ⅔ (mg/min) or less.

Item 3. The pharmaceutical composition according to Item 1 or 2, which is used such that 4 to 20 mg of the compound represented by Formula (1) or a metal salt thereof is transvascularly administered over a period of 10 minutes to 4 hours.

Item 4. The pharmaceutical composition according to any one of Items 1 to 3, which is for reducing a side effect on skin or subcutaneous tissue caused by administration of the compound represented by Formula (1) or a metal salt thereof.

Item 5. The pharmaceutical composition according to Item 4, wherein the side effect on skin or subcutaneous tissue caused by administration of the compound represented by Formula (1) or a metal salt thereof is at least one member selected from the group consisting of erythema, hyperhidrosis, and pruritus.

Item 6. The pharmaceutical composition according to any one of Items 1 to 5, which is for treatment of body fluid retention in heart failure (preferably congestive heart failure), body fluid retention in liver cirrhosis, hyponatremia due to the syndrome of inappropriate antidiuretic hormone secretion (SIADH), or autosomal dominant polycystic kidney disease.

Item 7. The pharmaceutical composition according to any one of Items 1 to 6, which is for patients in need of treatment by a single administration of 4 to 20 mg of the compound represented by Formula (1) or a metal salt thereof.

Item 8. The pharmaceutical composition according to any one of Items 1 to 7, which is a freeze-dried composition or an aqueous solution composition.

Item 9. The pharmaceutical composition according to any one of Items 1 to 8, wherein the metal salt is a disodium salt.

Item 10. A pharmaceutical formulation comprising the pharmaceutical composition of any one of Items 1 to 9 in a container (preferably a vial) such that the compound represented by Formula (1) or a metal salt thereof contained in the pharmaceutical composition is present in an amount of 4 to 20 mg.

### Advantageous Effects of Invention

A pharmaceutical composition comprising a specific tolvaptan prodrug and having reduced side effects is provided.

### DESCRIPTION OF EMBODIMENTS

The present disclosure preferably encompasses, for example, a pharmaceutical composition that comprises a specific tolvaptan prodrug, and that is used for administration at a specific rate; however, the disclosure is not limited thereto, and encompasses all of the matter disclosed herein and recognized by a person skilled in the art.

The pharmaceutical composition encompassed by the present disclosure comprises a compound represented by the following Formula (1):

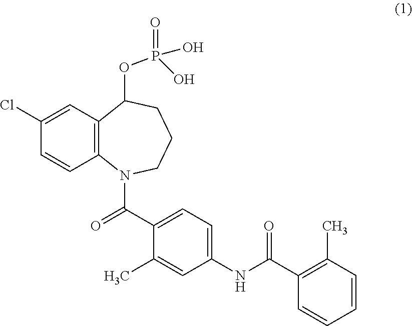 [SMILES: Cc1ccccc1C(=O)Nc1ccc(C(=O)N2CCCC(OP(=O)(O)O)c3cc(Cl)ccc32)c(C)c1]

or a metal salt thereof, and preferably comprises a metal salt of the compound represented by Formula (1). The compound represented by Formula (1) is sometimes referred to as “compound (1).” Further, a pharmaceutical composition comprising compound (1) or a salt thereof is sometimes referred to as “the composition according to the present disclosure.”

Compound (1) or a metal salt thereof serves as a specific tolvaptan prodrug contained in the composition according to the present disclosure. The specific tolvaptan prodrug is preferably a metal salt of compound (1).

The metal salt of compound (1) is preferably an alkali metal salt, an alkaline earth metal salt, or a zinc salt. More specifically, for example, a sodium salt (mono or disodium salt), a potassium salt (mono or dipotassium salt), a calcium salt, a magnesium salt, a zinc salt, and the like are preferable. Of these, a disodium salt is particularly preferable. The following is the structural formula of a disodium salt of compound (1).

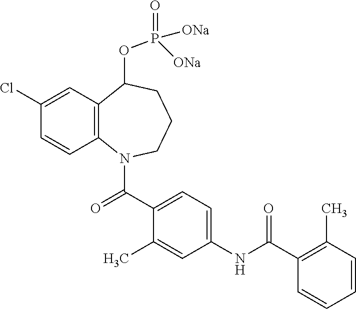

Compound (1) or a metal salt thereof can be produced by a known method, or a method easily conceivable of from a known method. For example, they can be produced by the method disclosed in PTL 1 (WO2007/074915) (in particular, the method disclosed in the Examples).

The composition according to the present disclosure is used such that 4 to 20 mg of compound (1) or a metal salt thereof is transvascularly administered over a period of 10 minutes or more. The transvascular administration is preferably intravenous administration. Since the composition according to the present disclosure is used for transvascular administration, preferable examples of the dosage form of the composition according to the present disclosure include injections, drip infusions, and the like. The upper or lower limit of the amount range of compound (1) or a metal salt thereof of 4 to 20 mg may be, for example, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, or 19.5 mg. For example, the range may be about 5 to 19 mg, about 6 to 18 mg, about 6.5 to 17.5 mg, about 7 to 17 mg, about 7.5 to 16.5 mg, or about 8 to 16 mg.

The time required for transvascular administration is preferably about 10 minutes to 4 hours. The upper or lower limit of the administration time range may be, for example, about 15 minutes, about 20 minutes, about 25 minutes, about 30 minutes, about 35 minutes, about 40 minutes, about 45 minutes, about 50 minutes, about 55 minutes, about 1 hour, about 1 hour and 5 minutes, about 1 hour and 10 minutes, about 1 hour and 15 minutes, about 1 hour and 20 minutes, about 1 hour and 25 minutes, about 1 hour and 30 minutes, about 1 hour and 35 minutes, about 1 hour and 40 minutes, about 1 hour and 45 minutes, about 1 hour and 50 minutes, about 1 hour and 55 minutes, about 2 hours, about 2 hours and 5 minutes, about 2 hours and 10 minutes, about 2 hours and 15 minutes, about 2 hours and 20 minutes, about 2 hours and 25 minutes, about 2 hours and 30 minutes, about 2 hours and 35 minutes, about 2 hours and 40 minutes, about 2 hours and 45 minutes, about 2 hours and 50 minutes, about 2 hours and 55 minutes, about 3 hours, about 3 hours and 5 minutes, about 3 hours and 10 minutes, about 3 hours and 15 minutes, about 3 hours and 20 minutes, about 3 hours and 25 minutes, about 3 hours and 30 minutes, about 3 hours and 35 minutes, about 3 hours and 40 minutes, about 3 hours and 45 minutes, about 3 hours and 50 minutes, or about 3 hours and 55 minutes. For example, the administration time range is preferably about 15 minutes to 4 hours or about 30 minutes to 4 hours, more preferably about 30 minutes to 3 hours or about 30 minutes to 2 hours, and even more preferably about 45 minutes to 2 hours or about 45 minutes to 1 hour and 30 minutes.

Further, the composition according to the present disclosure is preferably used such that the compound represented by Formula (1) or a metal salt thereof is administered at an average rate of ⅔ (mg/min) or less. That is, the administration is preferably performed at an average rate of ⅔ (mg/min) or less and greater than 0 (mg/min). The upper or lower limit of the range may be, for example, 1.5, 1.4, 1.3, 1.2, 1.1, 1, 0.9, 0.8, 0.7, 0.6, 0.5, 0.45, 0.4, 0.35, 0.3, 0.25, 0.2, 0.15, 0.1, 0.05, 0.04, 0.03, 0.02, or 0.01 (mg/min) on average. For example, the compound represented by Formula (1) or a metal salt thereof can be used for administration at an average rate of 1 to 0.05 (mg/min), 0.5 to 0.1 (mg/min), or 0.3 to 0.1 (mg/min).

Further, the composition according to the present disclosure can be preferably used for reducing side effects on the skin or subcutaneous tissue caused by administration of the compound represented by Formula (1) or a metal salt thereof. By administration of a specific tolvaptan prodrug, i.e., the compound represented by Formula (1) or a metal salt thereof, at a specific rate described above, it is possible to reduce side effects on skin or subcutaneous tissue that can be caused by the administration of tolvaptan or a prodrug thereof.

Examples of the side effects on skin or subcutaneous tissue include erythema, hyperhidrosis, and pruritus. According to the composition according to the present disclosure, one or more of these side effects on the skin or subcutaneous tissue can be reduced.

The composition according to the present disclosure can be used for the same usage as tolvaptan, and particularly preferably used for the same usage as the usage of known tolvaptan. For example, the composition according to the present disclosure is preferably used as a vasopressin receptor (in particular, V2 receptor) antagonist. More specifically, for example, the composition according to the present disclosure is preferably used for the treatment of body fluid retention in heart failure (preferably congestive heart failure), body fluid retention in liver cirrhosis, hyponatremia due to the syndrome of inappropriate antidiuretic hormone secretion (SIADH), or autosomal dominant polycystic kidney disease. The treatment of autosomal dominant polycystic kidney disease as used herein preferably refers to inhibition of an increase in kidney volume and/or inhibition of a decrease in kidney function in autosomal dominant polycystic kidney disease.

In particular, the composition according to the present disclosure can be preferably used to treat body fluid retention in heart failure for which the effect of other diuretics, such as loop diuretics, is insufficient; or body fluid retention in liver cirrhosis for which the effect of other diuretics, such as loop diuretics, is insufficient; or inhibit the progression of autosomal dominant polycystic kidney disease in which the kidney volume has already increased, and the rate of kidney volume increase is fast.

Further, the composition according to the present disclosure is preferably used for patients in need of treatment of the above diseases by a single administration of 4 to 20 mg of the compound represented by Formula (1) or a metal salt thereof. In particular, the composition according to the present disclosure is more preferably used for a single administration of the above amount to patients who suffer from side effects on the skin or subcutaneous tissue. The age of the subject of the administration of the composition according to the present disclosure is not particularly limited. For example, administration to an adult is preferable, and administration to an adult so as to satisfy the above conditions is more preferable.

The composition according to the present disclosure is preferably, but not particularly limited to, a freeze-dried composition or an aqueous solution composition comprising the compound represented by Formula (1) or a metal salt thereof.

The aqueous solution composition can be directly used for transvascular administration, and the transvascular administration is performed slowly over a period of time mentioned above (for example, over a period of 10 minutes or more, preferably 30 minutes or more, and more preferably about 60 minutes). Further, the aqueous solution composition may be used for transvascular administration after being dissolved in water (the water may contain other components known in the technical field of transvascular administration, preferably such as a physiological saline, a glucose injection solution, or various infusion formulations). The dosage form of the aqueous solution composition is not particularly limited. The aqueous solution composition can be administered by infusion from an infusion bag, a vial, or the like; or can be administered slowly by using an injection syringe. For more precise administration, the administration can be performed at a constant rate over a long period of time, for example, by using a device, such as an infusion pump or a syringe pump. Although not limited, the freeze-dried composition may be dissolved in water (the water may contain other components known in the technical field of transvascular administration, preferably such as a physiological saline, a glucose injection solution, or various infusion formulations) (i.e., constitution), and the obtained aqueous solution composition can be used for transvascular administration. Further, the aqueous solution composition obtained from the freeze-dried composition may further be diluted with water (the water may contain, for example, other components known in the technical field of transvascular administration, such as a physiological saline, a glucose injection solution, or various infusion formulations), and used for transvascular administration.

The freeze-dried composition or aqueous solution composition preferably comprises a disaccharide.

The disaccharide is preferably a disaccharide in which at least one of the two saccharides constituting the disaccharide is glucose. Specific examples include sucrose, maltose, trehalose, lactose, and the like, with sucrose being particularly preferable. The disaccharides can be used alone, or in a combination of two or more.

For example, the disaccharide is preferably present in an amount of preferably 0.5 to 70 parts by mass, more preferably 0.8 to 60 parts by mass, and still more preferably 1 to 15 parts by mass per part by mass of compound (1) or a metal salt thereof.

In particular, when the composition is a freeze-dried composition, the total amount of compound (1) or a metal salt thereof and a disaccharide is preferably 65% by mass or more, and more preferably 66, 67, 68, 69, or 70% by mass or more, of the entire composition.

The freeze-dried composition or the aqueous solution composition preferably further comprises a buffering agent. The buffering agent is preferably a phosphate buffering agent or a carbonate buffering agent, and particularly preferably a phosphate buffering agent. More specifically, for example, disodium hydrogen phosphate (sodium hydrogen phosphate hydrate) and/or sodium dihydrogen phosphate are preferable.

Further, the freeze-dried composition or the aqueous solution composition may optionally comprise a pH adjusting agent. In terms of the pH adjusting agent, specific examples of acidic pH adjusting agents include hydrochloric acid, acetic acid, phosphoric acid, and the like; and specific examples of basic pH adjusting agents include sodium hydroxide, potassium hydroxide, calcium carbonate, magnesium oxide, magnesium hydroxide, and the like.

In addition to the above, the freeze-dried composition or the aqueous solution composition may further optionally comprise a pharmaceutically acceptable carrier, in particular, a component known in the field of freeze-dried pharmaceutical formulations.

The composition according to the present disclosure (in particular, the freeze-dried composition or aqueous solution composition) is preferably sterile by sterilization or other methods. The sterilization method is not particularly limited. Examples include a method of performing aseptic filtration at the time of preparing the aqueous solution.

The compositions according to the present disclosure can be prepared based on a known method, for example, a method for preparing a freeze-dried pharmaceutical formulation. More specifically, in terms of the freeze-dried composition or aqueous solution composition, for example, an aqueous solution composition can be prepared by mixing compound (1) or a metal salt thereof and a disaccharide, and optionally a buffering agent, a pH adjusting agent, and the like, together with water for dissolution. Further, as described above, the freeze-dried composition can be prepared by freeze-drying the aqueous solution composition.

The present disclosure also encompasses a pharmaceutical formulation comprising an appropriate amount of the composition according to the present disclosure. Such a pharmaceutical formulation preferably comprises the composition according to the present disclosure in a container such that the compound represented by Formula (1) or a metal salt thereof contained in the composition is present in an amount of 4 to 20 mg. The pharmaceutical formulation is preferably used as a pharmaceutical formulation for a single administration or multiple administrations. The upper or lower limit of the amount range of compound (1) or a metal salt thereof of 4 to 20 mg may be, for example, 5, 6, 7, 8, 9, 10, 11, 12, 13, 14, 15, 16, 17, 18, or 19 mg. For example, the range may be about 5 to 19 mg, about 6 to 18 mg, about 7 to 17 mg, or about 8 to 16 mg. In particular, such a pharmaceutical formulation may be, for example, a vial product containing the freeze-dried composition (preferably a cake-like composition) or the aqueous solution composition only in the required amount mentioned above.

The terms “comprising” and “containing” include “consisting essentially of” and “consisting of.”

The various characteristics (e.g., properties, structures, and functions) described in each of the above embodiments may be combined in any way to specify the subject matter encompassed by the present disclosure.

### EXAMPLES

The subject matter encompassed by the present disclosure is described in more detail below. However, the subject matter is not limited to the following Examples.

### Production of Metal Salts of Compound (1)

Compound (1) and a disodium salt thereof were prepared according to the method disclosed in the Examples (in particular, Examples 1, 3, and 9) of PTL 1 (WO2007/074915). The disodium salt was used as compound A in the following analysis. The preparation was specifically performed as follows. In the description of the following specific preparation method, compound (1b) corresponds to compound (1), and the disodium salt of compound (1b) corresponds to compound A.

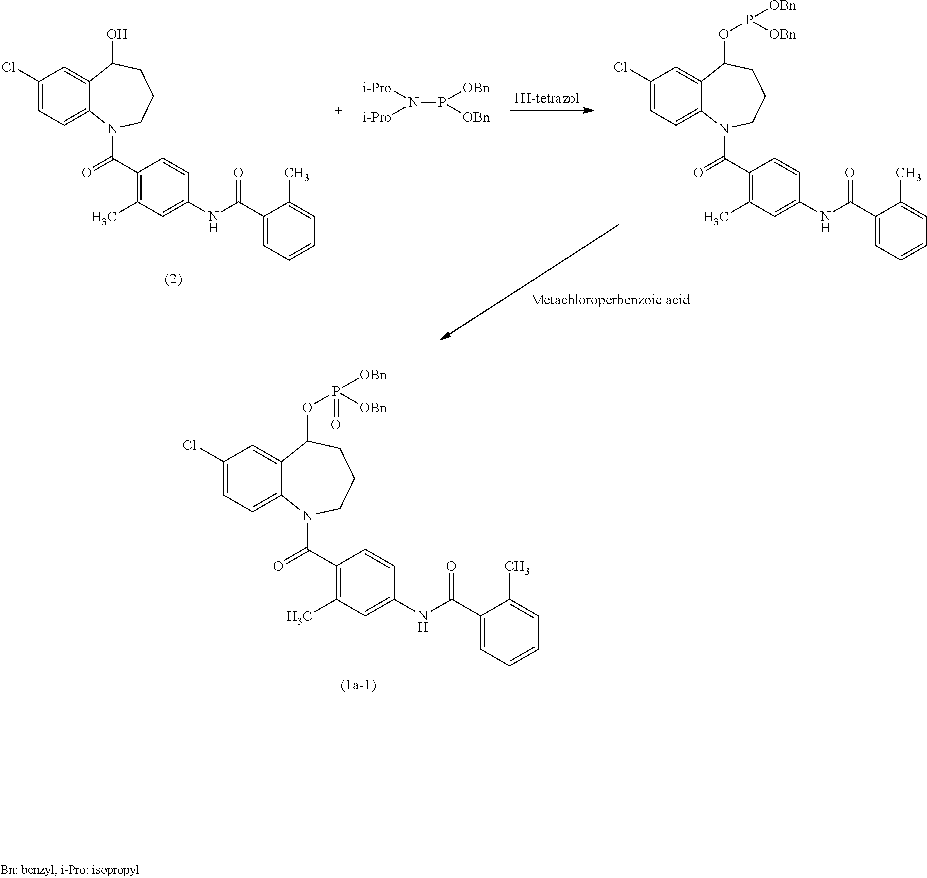

A 1.0 g quantity of tolvaptan and 460 mg of 1H-tetrazole were dissolved in 30 ml of methylene chloride; and 1.2 g of dibenzyl diisopropylphosphoramidite was added dropwise to this solution with stirring at room temperature, followed by stirring at the same temperature for 2 hours.

The obtained reaction mixture was cooled to −40° C., and a solution of 920 mg of metachloroperbenzoic acid in methylene chloride (6 ml) was added dropwise thereto. The mixture was stirred at the same temperature for 30 minutes, and at 0° C. for another 30 minutes. The reaction mixture was washed with an aqueous sodium thiosulfate solution and saturated aqueous sodium bicarbonate solution, and then dried over anhydrous sodium sulfate. The obtained reaction mixture was filtered and concentrated, and the residue was purified by silica gel column chromatography (eluent: n-hexane:ethyl acetate=1:1) to obtain 1.5 g of compound (1a-1) in an amorphous form (yield: 97.2%).

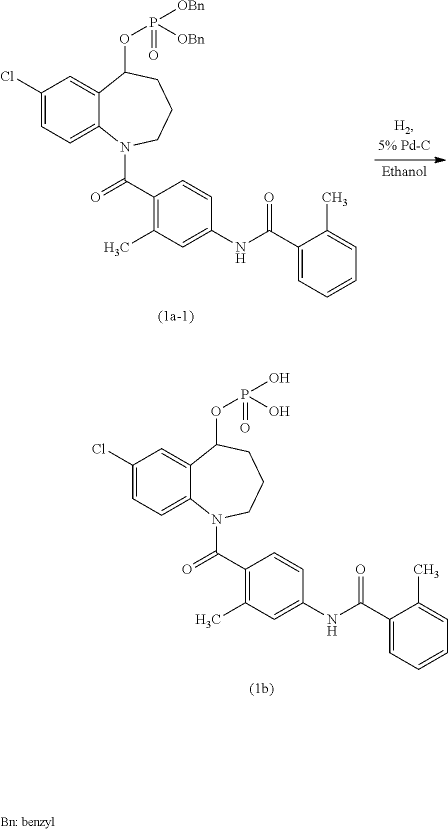 [SMILES: Cc1ccccc1C(=O)Nc1ccc(C(=O)N2CCCC(OP(=O)(OCc3ccccc3)OCc3ccccc3)c3cc(Cl)ccc32)c(C)c1 ; Cc1ccccc1C(=O)Nc1ccc(C(=O)N2CCCC(OP(=O)(O)O)c3cc(Cl)ccc32)c(C)c1]

A 5.3 g quantity of compound (1a-1) was dissolved in 100 ml of ethanol. Using 2 g of 5% palladium on carbon as a catalyst, the solution was catalytically reduced at ordinary temperature under ordinary pressure for 10 minutes. The catalyst was removed from the solution by filtration, and the obtained filtrate was concentrated (4.2 g). The obtained residue was crystallized from methanol-water. The crystals were collected by filtration, and dried under reduced pressure (diphosphorus pentoxide) to obtain 3.5 g of compound (1b) as a white powder (yield: 88.5%).

Further, 1.0 ml of 1N aqueous sodium hydroxide solution was added under ice-cooling to a solution of 276 mg (0.52 mmol) of compound (1b) in methanol (2 ml), and the resulting mixture was stirred for 5 minutes. The reaction mixture was concentrated under reduced pressure, and the residue was recrystallized from acetone-water to obtain 221 mg of disodium salt of compound (1b) as a white powder.

Additionally, according to the method disclosed in the Examples of PTL 1, a calcium salt, a magnesium salt, and a zinc salt of compound (1) were produced.

### Preparation of Freeze-Dried Formulations of Compound A

According to the compositions shown in Table 1 below, compound A, sucrose, sodium hydrogen phosphate hydrate, and sodium dihydrogen phosphate were dissolved in water for injection; and the pH was adjusted to 8.5 with sodium hydroxide, thus preparing aqueous solutions of the compositions shown in Table 1. After aseptic filtration, 5.21 mL of the aqueous solutions of the compositions were each filled into a sterilized glass vial. After being frozen to a temperature of −40° C. or lower, the vials were depressurized to vacuum, and the moisture content was removed by setting the shelf temperature to −10° C. Thereafter, the residual moisture content was removed by setting the shelf temperature to 30° C., thus obtaining aseptic freeze-dried compositions having the compositions shown in Table 2 (contained in a vial).

Table 1 shows the amount per mL, and Table 2 shows the amount per vial. The “25 mg formulation” contains 26.05 mg of the active ingredient (compound A) per vial.

TABLE 1
Aqueous solution compositions of compound A

| | Amount per mL (mg) | |
|---|---|---|
| | 0 mg | 25 mg |
| | formulation | formulation |
| Compound A | 0 | 5.0 |
| Sucrose | 55.0 | 48.0 |
| Sodium hydrogen phosphate hydrate | 18 | 18 |
| Sodium dihydrogen phosphate | 0.3 | 0.3 |

| | |
|---|---|
| Sodium hydroxide | q.s. to pH 8.5 |
| Water for injection | q.s. to pH 1.0 mL |

TABLE 2
Freeze-dried compositions of compound A

| | Amount per vial | |
|---|---|---|
| | (mg) | |
| | 0 mg | 25 mg |
| | formulation | formulation |
| Compound A | 0 | 26.05 |
| Sucrose | 286.55 | 250.08 |
| Sodium hydrogen phosphate hydrate | 93.78 | 93.78 |
| Sodium dihydrogen phosphate | 1.56 | 1.56 |

| | |
|---|---|
| Sodium hydroxide | q.s. |

Water for injection (5 mL) was added to each of the obtained 0 mg formulation and 25 mg formulation to dissolve the freeze-dried compositions (freeze-dried cakes), thus obtaining aqueous solutions for injection. When the 25 mg formulation was dissolved in 5 mL of water for injection, 5.21 mL of the aqueous solution composition (25 mg formulation (aqueous solution for injection)) shown in Table 1 comprising 5 mg/mL of the active ingredient (compound A) was reconstituted. Further, according to the compositions shown in Table 3 below, administration solutions were prepared by mixing the 25 mg formulation (aqueous solution for injection), the 0 mg formulation (aqueous solution for injection), and a physiological saline (physiological saline solution).

TABLE 3
Administration dose, administration solution, and administration time
in test of intravenous administration of compound A composition to healthy subjects

| | | | 25 mg formulation | 0 mg formulation | Physiological | Amount of |
|---|---|---|---|---|---|---|
| | | Administration dose | (aqueous solution | (aqueous solution | saline | administration |
| Administration | Administration | (amount of compound A) | for injection) | for injection) | solution | solution |
| solution | time | (mg) | (mL) | (mL) | (mL) | (mL) |
| 1 | 2 hours | 15 | 3 | 0 | 37 | 40 |
| 2 | | 7.5 | 1.5 | 1.5 | 37 | 40 |
| 3 | 5 minutes | 15 | 3 | 0 | 12 | 15 |
| 4 | | 7.5 | 1.5 | 1.5 | 12 | 15 |
| 5 | 1 minute | 15 | 3 | 0 | 0 | 3 |
| 6 | | 7.5 | 1.5 | 1.5 | 0 | 3 |

Each of the obtained administration solutions was intravenously administered with a syringe pump over the administration times shown in Table 3. The number of subjects to which each solution was administered was 6 healthy adult males (aged 20 to 40 years old) (N=6).

The number of subjects who developed, within 24 hours after the initiation of administration, side effects that could appear on the skin or subcutaneous tissue (erythema, hyperhidrosis, or pruritus) was recorded. Table 4 shows the results. When the administration solution containing 7.5 mg or 15 mg of compound A was intravenously administered over a period of 1 minute or 5 minutes, erythema, hyperhidrosis, or pruritus was frequently observed (Table 4: administration results of administration solutions 3 to 6). In contrast, when the administration solution containing 7.5 mg or 15 mg of compound A was slowly administered over a period of 2 hours, no side effects that could appear on the skin or subcutaneous tissue, such as erythema, hyperhidrosis, or pruritus, were observed (Table 4: administration results of administration solutions 1 and 2). Table 4 also shows the maximum blood concentration (Cₘₐₓ) of tolvaptan in the subjects to whom each of the administration solutions was administered.

TABLE 4
Side effects on the skin or subcutaneous tissue and maximum blood
concentration of tolvaptan in test of intravenous administration of compound A
composition to healthy subjects

| | | Administration dose | | | | Number of subjects | |
|---|---|---|---|---|---|---|---|
| | | (amount of | | | | who showed at least | |
| Administration | Administration | compound A) | Erythema | Hyperhidrosis | Pruritus | one symptom | Tolvaptan |
| solution | time | (mg) | (human) | (human) | (human) | (human) | Cmax (ng/mL) |
| 1 | 2 hours | 15 | 0 | 0 | 0 | 0 | 157.7 ± 23.98 |
| 2 | | 7.5 | 0 | 0 | 0 | 0 | 92.47 ± 20.81 |
| 3 | 5 minutes | 15 | 4 | 2 | 4 | 5 | 161.0 ± 26.12 |
| 4 | | 7.5 | 3 | 2 | 3 | 4 | 91.43 ± 26.39 |
| 5 | 1 minute | 15 | 5 | 2 | 4 | 5 | 168.8 ± 41.25 |
| 6 | | 7.5 | 5 | 0 | 4 | 5 | 99.60 ± 19.03 |

Formulation Examples 1 to 35 are shown below. Table 5 shows the amount (mg) per mL, and Table 6 shows the amount (mg) per vial.

TABLE 5

| | |
|---|---|
| 0 mg | 25 mg |
| formulation | formulation |

Formulation Example 15

| | | |
|---|---|---|
| Compound A | 0 | 5.0 |
| Trehalose | 82.1 | 78.7 |
| Glycine | 3.8 | 3.8 |

| | |
|---|---|
| Sodium hydroxide or hydrochloric acid | q.s. to pH 8.5 |
| Water for injection | q.s. to 1.0 mL |

Formulation Example 16

| | | |
|---|---|---|
| Compound A | 0 | 5.0 |
| D-glucose | 43.2 | 41.4 |
| Glycine | 3.8 | 3.8 |

| | |
|---|---|
| Sodium hydroxide or hydrochloric acid | q.s. to pH 8.5 |
| Water for injection | q.s. to 1.0 mL |

Formulation Example 17

| | | |
|---|---|---|
| Compound A | 0 | 5.0 |
| Lactose hydrate | 82.1 | 78.7 |
| Glycine | 3.8 | 3.8 |

| | |
|---|---|
| Sodium hydroxide or hydrochloric acid | q.s. to pH 8.5 |
| Water for injection | q.s. to 1.0 mL |

Formulation Example 18

| | | |
|---|---|---|
| Compound A | 0 | 5.0 |
| Sucrose | 82.1 | 78.7 |
| Glycine | 3.8 | 3.8 |

| | |
|---|---|
| Sodium hydroxide or hydrochloric acid | q.s. to pH 8.5 |
| Water for injection | q.s. to 1.0 mL |

Formulation Example 19

| | | |
|---|---|---|
| Compound A | 0 | 5.0 |
| Xylitol | 36.5 | 35.0 |
| Glycine | 3.8 | 3.8 |

| | |
|---|---|
| Sodium hydroxide or hydrochloric acid | q.s. to pH 8.5 |
| Water for injection | q.s. to 1.0 mL |

Formulation Example 20

| | | |
|---|---|---|
| Compound A | 0 | 5.0 |
| D-mannitol | 43.7 | 41.9 |
| Glycine | 3.8 | 3.8 |

| | |
|---|---|
| Sodium hydroxide or hydrochloric acid | q.s. to pH 8.5 |
| Water for injection | q.s. to 1.0 mL |

Formulation Example 21

| | | |
|---|---|---|
| Compound A | 0 | 5.0 |
| Sodium chloride | 7.0 | 6.7 |
| Glycine | 3.8 | 3.8 |

| | |
|---|---|
| Sodium hydroxide or hydrochloric acid | q.s. to pH 8.5 |
| Water for injection | q.s. to 1.0 mL |

Formulation Example 22

| | | |
|---|---|---|
| Compound A | 0 | 5.0 |
| Trehalose | 82.1 | 78.7 |
| Trometamol | 6.0 | 6.0 |

| | |
|---|---|
| Sodium hydroxide or hydrochloric acid | q.s. to pH 8.5 |
| Water for injection | q.s. to 1.0 mL |

Formulation Example 23

| | | |
|---|---|---|
| Compound A | 0 | 5.0 |
| D-glucose | 43.2 | 41.4 |
| Trometamol | 6.0 | 6.0 |

| | |
|---|---|
| Sodium hydroxide or hydrochloric acid | q.s. to pH 8.5 |
| Water for injection | q.s to 1.0 mL |

Formulation Example 24

| | | |
|---|---|---|
| Compound A | 0 | 5.0 |
| Lactose hydrate | 82.1 | 78.7 |
| Trometamol | 6.0 | 6.0 |

| | |
|---|---|
| Sodium hydroxide or hydrochloric acid | q.s to pH 8.5 |
| Water for injection | q.s. to 1.0 mL |

Formulation Example 25

| | | |
|---|---|---|
| Compound A | 0 | 5.0 |
| Sucrose | 82.1 | 78.7 |
| Trometamol | 6.0 | 6.0 |

| | |
|---|---|
| Sodium hydroxide or hydrochloric acid | q.s. to pH 8.5 |
| Water for injection | q.s. to 1.0 mL |

Formulation Example 26

| | | |
|---|---|---|
| Compound A | 0 | 5.0 |
| Xylitol | 36.5 | 35.0 |
| Trometamol | 6.0 | 6.0 |

| | |
|---|---|
| Sodium hydroxide or hydrochloric acid | q.s. to pH 8.5 |
| Water for injection | q.s. to 1.0 mL |

Formulation Example 27

| | | |
|---|---|---|
| Compound A | 0 | 5.0 |
| D-mannitol | 43.7 | 41.9 |
| Trometamol | 6.0 | 6.0 |

| | |
|---|---|
| Sodium hydroxide or hydrochloric acid | q.s. to pH 8.5 |
| Water for injection | q.s. to 1.0 mL |

Formulation Example 28

| | | |
|---|---|---|
| Compound A | 0 | 5.0 |
| Sodium chloride | 7.0 | 6.7 |
| Trometamol | 6.0 | 6.0 |

| | |
|---|---|
| Sodium hydroxide or hydrochloric acid | q.s. to pH 8.5 |
| Water for injection | q.s. to 1.0 mL |

Formulation Example 1

| | | |
|---|---|---|
| Compound A | 0 | 5.0 |
| Trehalose | 82.1 | 78.7 |
| Histidine | 7.8 | 7.8 |

| | |
|---|---|
| Sodium hydroxide or hydrochloric acid | q.s. to pH 8.5 |
| Water for injection | q.s. to 1.0 mL |

Formulation Example 2

| | | |
|---|---|---|
| Compound A | 0 | 5.0 |
| D-glucose | 43.2 | 41.4 |
| Histidine | 7.8 | 7.8 |

| | |
|---|---|
| Sodium hydroxide or hydrochloric acid | q.s. to pH 8.5 |
| Water for injection | q.s. to 1.0 mL |

Formulation Example 3

| | | |
|---|---|---|
| Compound A | 0 | 5.0 |
| Lactose hydrate | 82.1 | 78.7 |
| Histidine | 7.8 | 7.8 |

| | |
|---|---|
| Sodium hydroxide or hydrochloric acid | q.s. to pH 8.5 |
| Water for injection | q.s. to 1.0 mL |

Formulation Example 4

| | | |
|---|---|---|
| Compound A | 0 | 5.0 |
| Sucrose | 82.1 | 78.7 |
| Histdine | 7.8 | 7.8 |

| | |
|---|---|
| Sodium hydroxide or hydrochloric acid | q.s. to pH 8.5 |
| Water for injection | q.s. to 1.0 mL |

Formulation Example 5

| | | |
|---|---|---|
| Compound A | 0 | 5.0 |
| Xylitol | 36.5 | 35.0 |
| Histidine | 7.8 | 7.8 |

| | |
|---|---|
| Sodium hydroxide or hydrochloric acid | q.s. to pH 8.5 |
| Water for injection | q.s. to 1.0 mL |

Formulation Example 6

| | | |
|---|---|---|
| Compound A | 0 | 5.0 |
| D-mannitol | 43.7 | 41.9 |
| Histidine | 7.8 | 7.8 |

| | |
|---|---|
| Sodium hydroxide or hydrochloric acid | q.s. to pH 8.5 |
| Water for injection | q.s. to 1.0 mL |

Formulation Example 7

| | | |
|---|---|---|
| Compound A | 0 | 5.0 |
| Sodium chloride | 7.0 | 6.7 |
| Histidine | 7.8 | 7.8 |

| | |
|---|---|
| Sodium hydroxide or hydrochloric acid | q.s. to pH 8.5 |
| Water for injection | q.s. to 1.0 mL |

Formulation Example 8

| | | |
|---|---|---|
| Compound A | 0 | 5.0 |
| Trehalose | 82.1 | 78.7 |
| Sodium hydrogen carbonate | 4.2 | 4.2 |

| | |
|---|---|
| Sodium hydroxide or hydrochloric acid | q.s. to pH 8.5 |
| Water for injection | q.s. to 1.0 mL |

Formulation Example 9

| | | |
|---|---|---|
| Compound A | 0 | 5.0 |
| D-glucose | 43.2 | 41.4 |
| Sodium hydrogen carbonate | 4.2 | 4.2 |

| | |
|---|---|
| Sodium hydroxide or hydrochloric acid | q.s. to pH 8.5 |
| Water for injection | q.s. to 1.0 mL |

Formulation Example 10

| | | |
|---|---|---|
| Compound A | 0 | 5. 0 |
| Lactose hydrate | 82.1 | 78.7 |
| Sodium hydrogen carbonate | 4.2 | 4.2 |

| | |
|---|---|
| Sodium hydroxide or hydrochloric acid | q.s. to pH 8.5 |
| Water for injection | q.s. to 1.0 mL |

Formulation Example 12

| | | |
|---|---|---|
| Compound A | 0 | 5.0 |
| Sucrose | 82.1 | 78.7 |
| Sodium hydrogen carbonate | 4.2 | 4.2 |

| | |
|---|---|
| Sodium hydroxide or hydrochloric acid | q.s. to pH 8.5 |
| Water for injection | q.s. to 1.0 mL |

Formulation Example 12

| | | |
|---|---|---|
| Compound A | 0 | 5.0 |
| Xylitol | 36.5 | 35.0 |
| Sodium hydrogen carbonate | 4.2 | 4.2 |

| | |
|---|---|
| Sodium hydroxide or hydrochloric acid | q.s. to pH 8.5 |
| Water for injection | q.s. to 1.0 mL |

Formulation Example 13

| | | |
|---|---|---|
| Compound A | 0 | 5.0 |
| D-mannitol | 43.7 | 41.9 |
| Sodium hydrogen carbonate | 4.2 | 4.2 |

| | |
|---|---|
| Sodium hydroxide or hydrochloric acid | q.s. to pH 8.5 |
| Water for injection | q.s. to 1.0 mL |

Formulation Example 14

| | | |
|---|---|---|
| Compound A | 0 | 5.0 |
| Sodium chloride | 7.0 | 6.7 |
| Sodium hydrogen carbonate | 4.2 | 4.2 |

| | |
|---|---|
| Sodium hydroxide or hydrochloric acid | q.s. to pH 8.5 |
| Water for injection | q.s. to 1.0 mL |

Formulation Example 29

| | | |
|---|---|---|
| Compound A | 0 | 5.0 |
| Trehalose | 82.1 | 78.7 |
| Arginine | 8.7 | 8.7 |

| | |
|---|---|
| Sodium hydroxide or hydrochloric acid | q.s. to pH 8.5 |
| Water for injection | q.s. to 1.0 mL |

Formulation Example 30

| | | |
|---|---|---|
| Compound A | 0 | 5.0 |
| D-glucose | 43.2 | 41.4 |
| Arginine | 8.7 | 8.7 |

| | |
|---|---|
| Sodium hydroxide or hydrochloric acid | q.s. to pH 8.5 |
| Water for injection | q.s. to1.0 mL |

Formulation Example 31

| | | |
|---|---|---|
| Compound A | 0 | 5.0 |
| Lactose hydrate | 82.1 | 78.7 |
| Arginine | 8.7 | 8.7 |

| | |
|---|---|
| Sodium hydroxide or hydrochloric acid | q.s. to pH 8.5 |
| Water for injection | q.s. to 1.0 mL |

Formulation Example 32

| | | |
|---|---|---|
| Compound A | 0 | 5.0 |
| Sucrose | 82.1 | 78.7 |
| Arginine | 8.7 | 8.7 |

| | |
|---|---|
| Sodium hydroxide or hydrochloric acid | q.s. to pH 8.5 |
| Water for injection | q.s. to 1.0 mL |

Formulation Example 33

| | | |
|---|---|---|
| Compound A | 0 | 5.0 |
| Xylitol | 36.5 | 35.0 |
| Arginine | 8.7 | 8.7 |

| | |
|---|---|
| Sodium hydroxide or hydrochloric acid | q.s. to pH 8.5 |
| Water for injection | q.s. to 1.0 mL |

Formulation Example 34

| | | |
|---|---|---|
| Compound A | 0 | 5.0 |
| D-mannitol | 43.7 | 41.9 |
| Arginine | 8.7 | 8.7 |

| | |
|---|---|
| Sodium hydroxide or hydrochloric acid | q.s. to pH 8.5 |
| Water for injection | q.s. to 1.0 mL |

Formulation Example 35

| | | |
|---|---|---|
| Compound A | 0 | 5.0 |
| Sodium chloride | 7.0 | 6.7 |
| Arginine | 8.7 | 8.7 |

| | |
|---|---|
| Sodium hydroxide or hydrochloric acid | q.s. to pH 8.5 |
| Water for injection | q.s. to 1.0 mL |

TABLE 6

| | |
|---|---|
| 0 mg | 25 mg |
| formulation | formulation |

Formulation Example 1

| | | |
|---|---|---|
| Compound A | 0 | 26.05 |
| Trehalase | 427.7 | 410.0 |
| Histidine | 40.6 | 40.6 |

| | |
|---|---|
| Sodium hydroxide or hydrochloric acid | q.s. to pH 8.5 |
| Water for injection | q.s. to 5.21 mL |

Formulation Example 2

| | | |
|---|---|---|
| Compound A | 0 | 26.05 |
| D-glucose | 225.1 | 215.7 |
| Histidine | 40.6 | 40.6 |

| | |
|---|---|
| Sodium hydroxide or hydrochloric acid | q.s. to pH 8.5 |
| Water for injection | q.s. to 5.21 mL |

Formulation Example 3

| | | |
|---|---|---|
| Compound A | 0 | 26.05 |
| Lactose hydrate | 427.7 | 410.0 |
| Histidine | 40.6 | 40.6 |

| | |
|---|---|
| Sodium hydroxide or hydrochloric acid | q.s. to pH 8.5 |
| Water for injection | q.s. to 5.21 mL |

Formulation Example 4

| | | |
|---|---|---|
| Compound A | 0 | 26.05 |
| Sucrose | 427.7 | 410.0 |
| Histidine | 40.6 | 40.6 |

| | |
|---|---|
| Sodium hydroxide or hydrochloric acid | q.s. to pH 8.5 |
| Water for injection | q.s. to 5.21 mL |

Formulation Example 5

| | | |
|---|---|---|
| Compound A | 0 | 26.05 |
| Xylitol | 190.2 | 182.4 |
| Histidine | 40.6 | 40.6 |

| | |
|---|---|
| Sodium hydroxide or hydrochloric acid | q.s. to pH 8.5 |
| Water for injection | q.s. to 5.21 mL |

Formulation Example 6

| | | |
|---|---|---|
| Compound A | 0 | 26.05 |
| D-mannitol | 227.7 | 218.3 |
| Histidine | 40.6 | 40.6 |

| | |
|---|---|
| Sodium hydroxide or hydrochloric acid | q.s. to pH 8.5 |
| Water for injection | q.s. to 5.21 mL |

Formulation Example 7

| | | |
|---|---|---|
| Compound A | 0 | 26.05 |
| Sodium chloride | 36.5 | 34.9 |
| Histidine | 40.6 | 40.6 |

| | |
|---|---|
| Sodium hydroxide or hydrochloric acid | q.s. to pH 8.5 |
| Water for injection | q.s. to 5.21 mL |

Formulation Example 8

| | | |
|---|---|---|
| Compound A | 0 | 26.05 |
| Trehalose | 427.7 | 410.0 |
| Sodium hydrogen carbonate | 21.9 | 21.9 |

| | |
|---|---|
| Sodium hydroxide or hydrochloric acid | q.s. to pH 8.5 |
| Water for injection | q.s. to 5.21 mL |

Formulation Example 9

| | | |
|---|---|---|
| Compound A | 0 | 26.05 |
| D-glucose | 225.1 | 215.7 |
| Sodium hydrogen carbonate | 21.9 | 21.9 |

| | |
|---|---|
| Sodium hydroxide or hydrochloric acid | q.s. to pH 8.5 |
| Water for injection | q.s. to 5.21 mL |

Formulation Example 10

| | | |
|---|---|---|
| Compound A | 0 | 26.05 |
| Lactose hydrate | 427.7 | 410.0 |
| Sodium hydrogen carbonate | 21.9 | 21.9 |

| | |
|---|---|
| Sodium hydroxide or hydrochloric acid | q.s. to pH 8.5 |
| Water for injection | q.s. to 5.21 mL |

Formulation Example 12

| | | |
|---|---|---|
| Compound A | 0 | 26.05 |
| Sucrose | 427.7 | 410.0 |
| Sodium hydrogen carbonate | 21.9 | 21.9 |

| | |
|---|---|
| Sodium hydroxide or hydrochloric acid | q.s. to pH 8.5 |
| Water for injection | q.s. to 5.21 mL |

Formulation Example 12

| | | |
|---|---|---|
| Compound A | 0 | 26.05 |
| Xylitol | 190.2 | 182.4 |
| Sodium hydrogen carbonate | 21.9 | 21.9 |

| | |
|---|---|
| Sodium hydroxide or hydrochloric acid | q.s. to pH 8.5 |
| Water for injection | q.s. to 5.21 mL |

Formulation Example 13

| | | |
|---|---|---|
| Compound A | 0 | 26.05 |
| D-mannitol | 227.7 | 218.3 |
| Sodium hydrogen carbonate | 21.9 | 21.9 |

| | |
|---|---|
| Sodium hydroxide or hydrochloric acid | q.s. to pH 8.5 |
| Water for injection | q.s. to 5.21 mL |

Formulation Example 14

| | | |
|---|---|---|
| Compound A | 0 | 26.05 |
| Sodium chloride | 36.5 | 34.9 |
| Sodium hydrogen carbonate | 21.9 | 21.9 |

| | |
|---|---|
| Sodium hydroxide or hydrochloric acid | q.s. to pH 8.5 |
| Water for injection | q.s. to 5.21 mL |

Formulation Example 15

| | | |
|---|---|---|
| Compound A | 0 | 26.05 |
| Trehalose | 427.7 | 410.0 |
| Glycine | 19.8 | 19.8 |

| | |
|---|---|
| Sodium hydroxide or hydrochloric acid | q.s. to pH 8.5 |
| Water for injection | q.s. to 5.21 mL |

Forrnulation Example 16

| | | |
|---|---|---|
| Compound A | 0 | 26.05 |
| D-glucose | 225.1 | 215.7 |
| Glycine | 19.8 | 19.8 |

| | |
|---|---|
| Sodium hydroxide or hydrochloric acid | q.s. to pH 8.5 |
| Water for injection | q.s. to 5.21 mL |

Formulation Example 17

| | | |
|---|---|---|
| Compound A | 0 | 26.05 |
| Lactose hydrate | 427.7 | 410.0 |
| Glycine | 19.8 | 19.8 |

| | |
|---|---|
| Sodium hydroxide or hydrochloric acid | q.s. to pH 8.5 |
| Water for injection | q.s. to 5.21 mL |

Formulation Example 18

| | | |
|---|---|---|
| Compound A | 0 | 26.05 |
| Sucrose | 427.7 | 410.0 |
| Glycine | 19.8 | 19.8 |

| | |
|---|---|
| Sodium hydroxide or hydrochloric acid | q.s. to pH 8.5 |
| Water for injection | q.s. to 5.21 mL |

Formlation Example 19

| | | |
|---|---|---|
| Compound A | 0 | 26.05 |
| Xylitol | 190.2 | 182.4 |
| Glycine | 19.8 | 19.5 |

| | |
|---|---|
| Sodium hydroxide or hydrochloric acid | q.s. to pH 8.5 |
| Water for injection | q.s. to 5.21 mL |

Formulation Example 20

| | | |
|---|---|---|
| Compound A | 0 | 26.05 |
| D-mannitol | 227.7 | 218.3 |
| Glycine | 19.8 | 19.8 |

| | |
|---|---|
| Sodium hydroxide or hydrochloric acid | q.s. to pH 8.5 |
| Water for injection | q.s. to 5.21 mL |

Formulation Example 21

| | | |
|---|---|---|
| Compound A | 0 | 26.05 |
| Sodium chloride | 36.5 | 34.9 |
| Glycine | 19.8 | 19.8 |

| | |
|---|---|
| Sodium hydroxide or hydrochloric acid | q.s. to pH 8.5 |
| Water for injection | q.s. to 5.21 mL |

Formulation Example 22

| | | |
|---|---|---|
| Compound A | 0 | 26.05 |
| Trehalose | 427.7 | 410.0 |
| Trometamol | 31.3 | 31.3 |

| | |
|---|---|
| Sodium hydroxide or hydrochloric acid | q.s. to pH 8.5 |
| Water for injection | q.s. to 5.21 mL |

Formulation Example 23

| | | |
|---|---|---|
| Compound A | 0 | 26.05 |
| D-glucose | 225.1 | 215.7 |
| Trometamol | 31.3 | 31.3 |

| | |
|---|---|
| Sodium hydroxide or hydrochloric acid | q.s. to pH 8.5 |
| Water for injection | q.s. to 5.21 mL |

Formulation Example 24

| | | |
|---|---|---|
| Compound A | 0 | 26.05 |
| Lactose hydrate | 427.7 | 410.0 |
| Trometamol | 31.3 | 31.3 |

| | |
|---|---|
| Sodium hydroxide or hydrochloric acid | q.s. to pH 8.5 |
| Water for injection | q.s. to 5.21 mL |

Formulation Example 25

| | | |
|---|---|---|
| Compound A | 0 | 26.05 |
| Sucrose | 82.1 | 78.7 |
| Trometamol | 31.3 | 31.3 |

| | |
|---|---|
| Sodium hydroxide or hydrochloric acid | q.s. to pH 8.5 |
| Water for injection | q.s. to 5.21 mL |

Formulation Example 26

| | | |
|---|---|---|
| Compound A | 0 | 26.05 |
| Xylitol | 190.2 | 182.4 |
| Trometamol | 31.3 | 31.3 |

| | |
|---|---|
| Sodium hydroxide or hydrochloric acid | q.s. to pH 8.5 |
| Water for injection | q.s. to 5.21 mL |

Formulation Example 27

| | | |
|---|---|---|
| Compound A | 0 | 26.05 |
| D-mannitol | 227 7 | 218.3 |
| Trometamol | 31.3 | 31.3 |

| | |
|---|---|
| Sodium hydroxide or hydrochloric acid | q.s. to pH 8.5 |
| Water for injection | q.s. to 5.21 mL |

Formulation Example 28

| | | |
|---|---|---|
| Compound A | 0 | 26.05 |
| Sodium chloride | 36.5 | 34.9 |
| Trometamol | 31.3 | 31.3 |

| | |
|---|---|
| Sodium hydroxide or hydrochloric acid | q.s. to pH 8.5 |
| Water for injection | q.s. to 5.21 mL |

Formulation Example 29

| | | |
|---|---|---|
| Compound A | 0 | 26.05 |
| Trehalose | 427.7 | 410.0 |
| Arginine | 45.3 | 45.3 |

| | |
|---|---|
| Sodium hydroxide or hydrochloric acid | q.s. to pH 8.5 |
| Water for injection | q.s. to 5.21 mL |

Formulation Example 30

| | | |
|---|---|---|
| Compound A | 0 | 26.05 |
| D-glucose | 225.1 | 215.7 |
| Arginine | 45.3 | 45.3 |

| | |
|---|---|
| Sodium hydroxide or hydrochloric acid | q.s. to pH 8.5 |
| Water for injection | q.s. to 5.21 mL |

Formulation Example 31

| | | |
|---|---|---|
| Compound A | 0 | 26.05 |
| Lactose hydrate | 427.7 | 410.0 |
| Arginine | 45.3 | 45.3 |

| | |
|---|---|
| Sodium hydroxide or hydrochloric acid | q.s. to pH 8.5 |
| Water for injection | q.s. to 5.21 mL |

Formulation Example 32

| | | |
|---|---|---|
| Compound A | 0 | 26.05 |
| Sucrose | 82.1 | 78.7 |
| Arginine | 45.3 | 45.3 |

| | |
|---|---|
| Sodium hydroxide or hydrochloric acid | q.s. to pH 8.5 |
| Water for injection | q.s. to 5.21 mL |

Formulation Example 33

| | | |
|---|---|---|
| Compound A | 0 | 26.05 |
| Xylitol | 190.2 | 182.4 |
| Arginine | 45.3 | 45.3 |

| | |
|---|---|
| Sodium hydroxide or hydrochloric acid | q.s. to pH 8.5 |
| Water for injection | q.s. to 5.21 mL |

Formulation Example 34

| | | |
|---|---|---|
| Compound A | 0 | 26.05 |
| D-mannitol | 227.7 | 218.3 |
| Arginine | 45.3 | 45.3 |

| | |
|---|---|
| Sodium hydroxide or hydrochloric acid | q.s. to pH 8.5 |
| Water for injection | q.s. to 5.21 mL |

Formulation Example 35

| | | |
|---|---|---|
| Compound A | 0 | 26.05 |
| Sodium chloride | 36.5 | 34.9 |
| Arginine | 45.3 | 45.3 |

| | |
|---|---|
| Sodium hydroxide or hydrochloric acid | q.s. to pH 8.5 |
| Water for injection | q.s. to 5.21 mL |

1. A pharmaceutical composition comprising a compound represented by Formula (1): 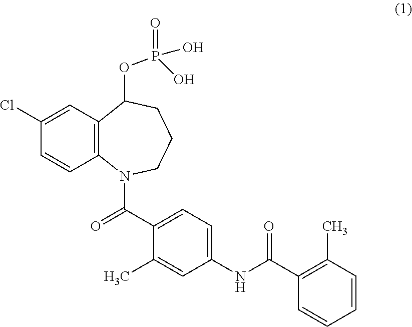 [SMILES: Cc1ccccc1C(=O)Nc1ccc(C(=O)N2CCCC(OP(=O)(O)O)c3cc(Cl)ccc32)c(C)c1]
or a metal salt thereof,
wherein the pharmaceutical composition is used such that 4 to 20 mg of the compound represented by Formula (1) or a metal salt thereof is transvascularly administered over a period of 10 minutes or more.

2. The pharmaceutical composition according to claim 1, which is used such that the compound represented by Formula (1) or a metal salt thereof is administered at an average rate of ⅔ (mg/min) or less.

3. The pharmaceutical composition according to claim 1, which is used such that 4 to 20 mg of the compound represented by Formula (1) or a metal salt thereof is transvascularly administered over a period of 10 minutes to 4 hours.

4. The pharmaceutical composition according to claim 1, which is for reducing a side effect on skin or subcutaneous tissue caused by administration of the compound represented by Formula (1) or a metal salt thereof.

5. The pharmaceutical composition according to claim 4, wherein the side effect on skin or subcutaneous tissue caused by administration of the compound represented by Formula (1) or a metal salt thereof is at least one member selected from the group consisting of erythema, hyperhidrosis, and pruritus.

6. The pharmaceutical composition according to claim 1, which is for treatment of body fluid retention in heart failure, body fluid retention in liver cirrhosis, hyponatremia due to the syndrome of inappropriate antidiuretic hormone secretion (SIADH), or autosomal dominant polycystic kidney disease.

7. The pharmaceutical composition according to claim 1, which is for patients in need of treatment by a single administration of 4 to 20 mg of the compound represented by Formula (1) or a metal salt thereof.

8. The pharmaceutical composition according to claim 1, which is a freeze-dried composition or an aqueous solution composition.

9. The pharmaceutical composition according to claim 1, wherein the metal salt is a disodium salt.

10. A pharmaceutical formulation comprising the pharmaceutical composition of claim 1 in a container such that the compound represented by Formula (1) or a metal salt thereof contained in the pharmaceutical composition is present in an amount of 4 to 20 mg.
